(19) United States
(12) Patent Application Publication
(10) Pub. No.: US 2018/0028776 A1
(43) Pub. Date: Feb. 1, 2018

(54) AIR TREATMENT SYSTEMS AND DEVICES

(71) Applicant: Ken E Clark, Wailuku, HI (US)

(72) Inventor: Ken E Clark, Wailuku, HI (US)

(21) Appl. No.: 15/223,322

(22) Filed: Jul. 29, 2016

(51) Int. Cl. A61M 21/02; B65D 65/02; A61L 9/12

(52) U.S. Cl. CPC A61M 21/02; A61L 9/12; B65D 65/02; A61L 2209/15; A61M 2021/0016

(57) ABSTRACT

An air treatment device can include a pad having a front side and a back side that faces opposite the front side. An aromatic scent can be infused within the pad. Additionally, an adhesive layer can be disposed along the back side of the pad that can allow the pad to adhere to textile materials, such as pillow cases and clothing.

### BACKGROUND

### Field

Various embodiments relate to pads. Certain embodiments relate to scented pads.

### Description of Related Art

Aromatherapy can have many benefits that enhance a person's well-being. For example, aromatherapy can ease depression, boost energy levels, speed up the healing process, eliminate headaches, boost cognitive performance, induce sleep, strengthen the immune system, reduce pain, improve digestion, act as a decongestant, help with respiratory issues, open nasal passages, act as a mental stimulant, helps with insomnia, eases stress and anxiety and increase circulation.

There currently are electric plug-in machines such as humidifiers, vaporizers, steam inhalers and battery operated diffusers that emit aroma. Additionally, scent strips can incorporate fragrances onto paper for sampling purposes. However, these systems have shortcomings. The electrical plug-in machines are stationary devices meant to be used in homes and businesses. The diffusers use batteries that need constant replacing and are only suitable for a home or office setting. Additionally, the scent strips are mainly used for sampling purposes and not long-term use. As a result, there is a need for devices and systems that provide the benefits of aromatherapy while overcoming the shortcomings of the systems known within the prior art.

### SUMMARY

The disclosure includes an air treatment device comprising a pad having a front side and a back side that faces opposite the front side. The air treatment device can include an aromatic scent infused within the pad, and an adhesive layer disposed along the back side of the pad. Additionally, the air treatment device can be enclosed within a package. In some embodiments, the air treatment device comprises a porous flexible hypoallergenic fabric type material base (hereafter called “base”), a pressure sensitive latex-free acrylic adhesive layer (hereafter called “adhesive layer”) disposed in a strip along the edges of the backside of the base that allows the product to adhere to a surface for use; and a smaller aromatic scent infused pad (hereafter called “pad”) having a front side and a back side that faces opposite the front side. The front side of the pad is centered onto the adhesive layer on the base so that the pad is secured to the base at the pad's edges, but the center is unobstructed by adhesive. A porous film cover attaches to the back side of the pad, thereby protecting against seepage, leakage, or transfer of the scent infusion material. And finally, a two-part removable release paper, which is the same size as the backing; which adheres to the backing via the remainder of the exposed adhesive layer to cover and protect the pad; and which, when removed, allows attachment of the air treatment device to an appropriate surface for use. Additionally, the air treatment device can be enclosed within a package.

In some embodiments, the air treatment device includes an adhesive layer that is arranged and configured to adhere to a textile material for at least a predetermined amount of time. For instance, this predetermined amount of time can be at least 12 hours.

Furthermore, in some embodiments, the aromatic scent is arranged and configured to penetrate through the pad and textile material whereby the aromatic scent permeates through the surrounding air. The aromatic scent can define a chemical state comprising at least one of a gel, liquid, paste, and powder. Additionally, the aromatic scent can define a scent type that can comprise at least one of coffee, menthol, eucalyptus, peppermint, lavender, camphor, jasmine, sandalwood, lilac, lily, chamomile, vanilla, tea tree, ylang ylang, rose, clove, cedarwood, bergamot, clementine, frankincense, lemon, marjoram, patchouli, rosemary, blue cypress, and cocoa.

Even still, in some embodiments, a porous film cover can be attached to the front side of the pad. The porous film cover can be arranged and configured to seal at least a portion of the aromatic scent infused within the pad.

In still another embodiment, a release paper can be removably attached to the adhesive layer. The adhesive layer can comprise a latex-free acrylic adhesive. Also, the release paper can then be arranged and configured to protect the adhesive layer to thereby preserve an ability of the adhesive layer to adhere to the textile material.

Additionally, in some embodiments, an outer package can comprise at least one of foil and/or plastic. The outer package can contain a first internal package and a second internal package.

The textile material can comprise at least one of a plant textile, a synthetic textile, and an animal textile. Further, the textile material can also comprise one of clothing and/or linens such as bed sheets, a pillow, and a pillow case.

Even still, in another embodiment, the pad can comprise at least one of micro-porous flexible hypoallergenic fabric and plastic. Specifically, the pad can be a micro-porous flexible hypoallergenic fabric or a micro-porous flexible hypoallergenic plastic.

The disclosure also includes an air treatment system that can comprise a first scented device including a first pad having a front side and a back side that faces opposite the front side. Additionally, the air treatment system can include a first aromatic scent infused within the first pad, a first adhesive layer disposed along the back side of the first pad, and a first release paper removably attached to the first adhesive layer. The system can include a first porous film cover attached to the front side of the first pad, and a first internal package that encloses the first scented device can be included in the air treatment system. The first adhesive layer can be arranged and configured to adhere to a textile material for at least a predetermined amount of time.

The air treatment system can also include a second scented device including a second pad having a front side and a back side that faces opposite the front side, a second aromatic scent infused within the second pad. The air treatment system can comprise a second adhesive layer disposed along the back side of the second pad, a second release paper removably attached to the second adhesive layer, a second porous film cover attached to the front side of the second pad, and a second internal package that encloses the second scented device. The second adhesive layer can be arranged and configured to adhere to the textile material for at least the predetermined amount of time.

In some embodiments, the system includes an outer package that encloses the first internal package and the second internal package. Furthermore, the first aromatic scent and the second aromatic scent can be the same. Alternatively, the first aromatic scent and the second aromatic scent can be different.

### BRIEF DESCRIPTION OF THE DRAWINGS

These and other features, aspects, and advantages are described below with reference to the drawings, which are intended to illustrate, but not to limit, the invention. In the drawings, like reference characters denote corresponding features consistently throughout similar embodiments.

FIG. 1 illustrates a front side view of an air treatment device, according to some embodiments.

FIGS. 2, 3, and 4 illustrate back side views of air treatment devices, according to some embodiments.

FIG. 5 illustrates a perspective view of an air treatment device attached to a textile material, according to some embodiments.

FIG. 6 illustrates a cross-sectional view of an air treatment device, according to some embodiments.

FIG. 7 illustrates a view of an air treatment device enclosed within a package, according to some embodiments.

FIG. 8 illustrates a view of an air treatment system, according to some embodiments.

### DETAILED DESCRIPTION OF THE DRAWINGS

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### Introduction

The present invention describes an adhesive pad with an infused scent. The aroma from the pad can be emitted into the surrounding air. In some embodiments, the scented adhesive pad comprises a micro-porous flexible hypoallergenic fabric type material, a latex-free acrylic adhesive layer, a scent infused pad centered on the adhesive layer fabric type material, a perforated plastic cover over the scent infused pad, a wax paper type covering over the adhesive, and an outer foil package to enclose the pad.

Additionally, a system for using an adhesive micro-porous flexible hypoallergenic fabric type material that is air and vapor permeable is also described by the present invention. The scent infused pad can comprise a thin layer of latex-free acrylic adhesive for adhering to any type of fabric. The scent infused pad can also incorporate a fragrance, chemically compounded fragrance, or any other type of fragrance, such as a scented essential oil or any other type of scented oil, extract, synthetic equivalent, or a mixture of any scents or fragrances that are infused into the pad centered onto the fabric-type material. This method is not just intended to be a great scent-aid type pad that is worn on clothing such as shirts, blouses, dresses, jackets, or any other wearable garment top, but can come in larger sizes for use on linens, such as bed sheets, pillows, and pillow cases. The scent infused pad will not ooze, bleed out, run or stain any cloth and/or fabric type textile materials.

The foregoing and other objects, features and advantages of the invention will become more readily apparent from the following detailed description of the preferred embodiment of the invention that proceeds with reference to the accompanying drawings.

### Air Treatment Device Embodiments

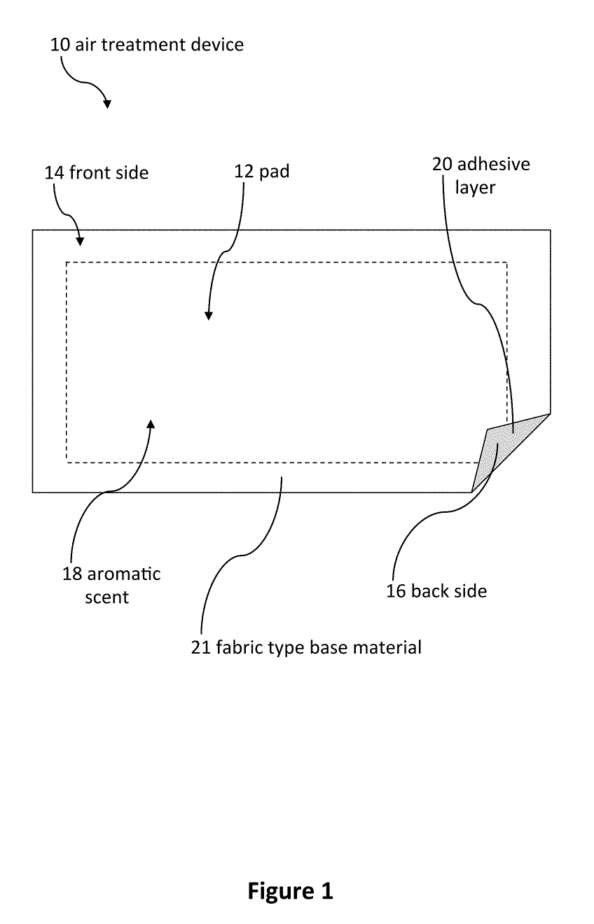

FIG. 1 illustrates an air treatment device 10 that provides the benefits of aromatherapy while overcoming the shortcomings of the systems known within the prior art. The front side 14 of the air treatment device 10 can include a fabric type base material 21 that runs continuously along the entire front side 14. The back side 16 of the air treatment device 10 can comprise a pad 12 (presented in broken lines), an aromatic scent 18 infused within the pad 12, and an adhesive layer 20 disposed along the outer edges of the pad 12. In some embodiments, the adhesive layer 20 can be composed of latex-free acrylic material.

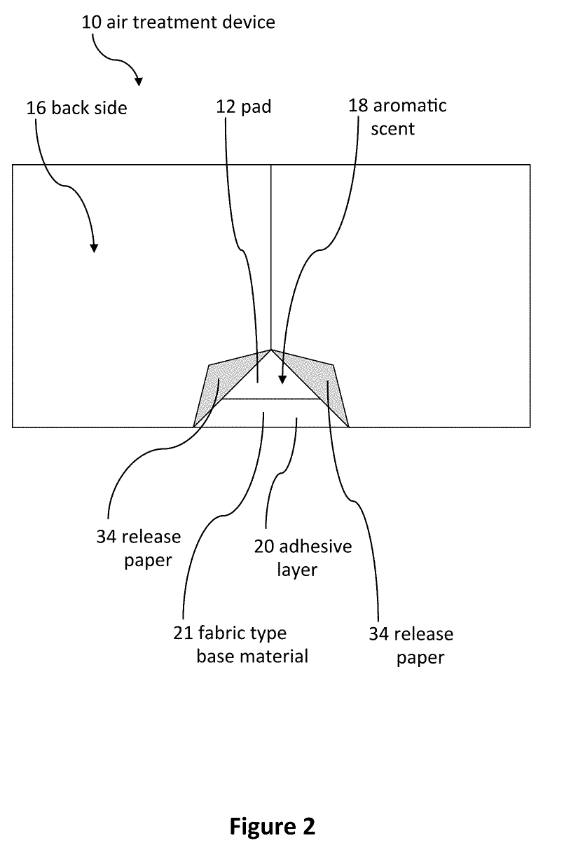

FIG. 2 illustrates a back side view of an air treatment device 10. In some embodiments, the back side 16 of the air treatment device 10 comprises a porous flexible hypoallergenic fabric type base material 21, a pressure sensitive latex-free acrylic adhesive layer 20 disposed in a strip along the edges of the back side 16 of the base 21 that allows the product to adhere to a surface for use. The air treatment device 10 may include a smaller pad 12 having an aromatic scent 18 infused within the pad 12. The pad 12 can be centered onto the adhesive layer 20 on the base 21 so that the pad 12 is secured to the base 21 at the pad's edges. As such, the center of the pad 12 may be unobstructed by adhesive 20.

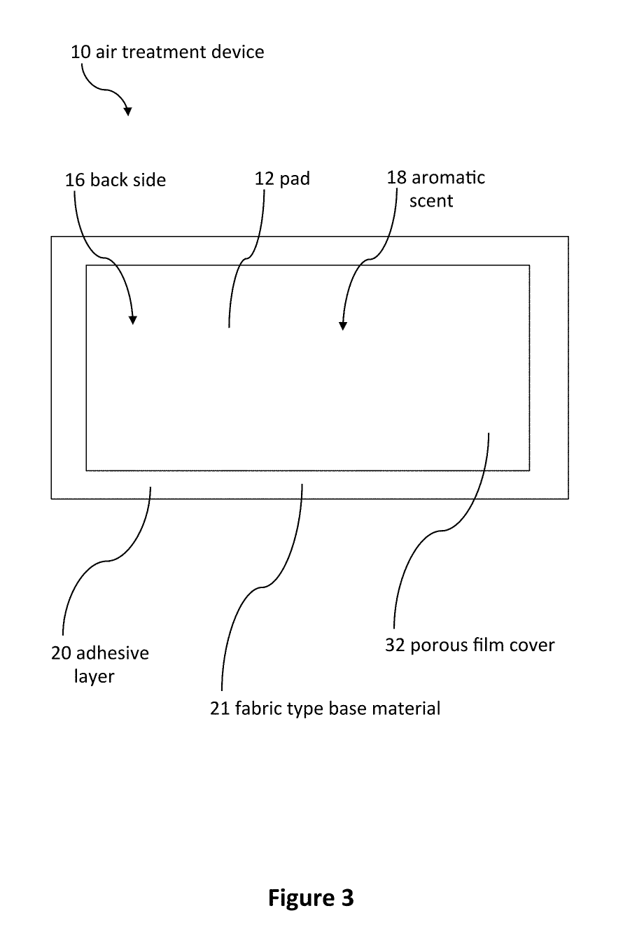

As shown in FIGS. 2 and 3, in some embodiments, the air treatment device 10 may include release paper 34 that is disposed along the back side 16 over the top of the pad 12, the adhesive layer 20, and the fabric type base material 21. The release paper 34 may be peeled back (and discarded) to thereby expose the pad 12, the adhesive layer 20, and the fabric type base material 21. In this regard, once the release paper 34 is removed, the aromatic scent 18 may permeate throughout the air and the air treatment device 10 may be attached to an appropriate surface. The release paper 34 may be a one or two-part removable release paper, which is the same size as the backing. In this regard, the release paper 34 can adhere to the back side 16 via the remainder (or edges) of the exposed adhesive layer 20 to cover and protect the pad.

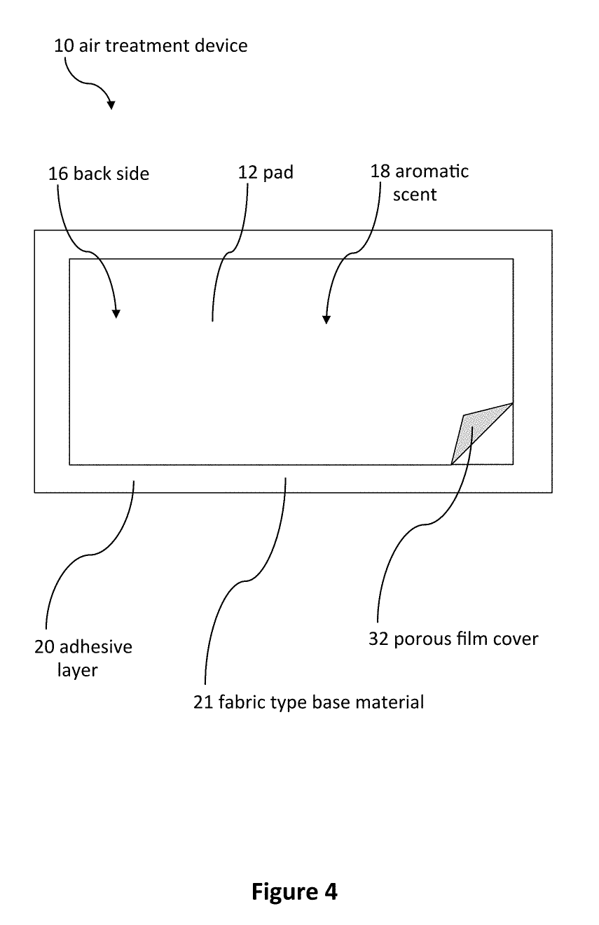

Now with reference to FIGS. 3 and 4, the air treatment device 10 may include a porous film cover 32 that attaches to the back side 16 of the air treatment device 10. The porous film cover 32 may protect against seepage, leakage, or transfer of the scent infusion material. As shown in FIG. 4, the porous film cover 32 may be peeled back and removed from the pad 12.

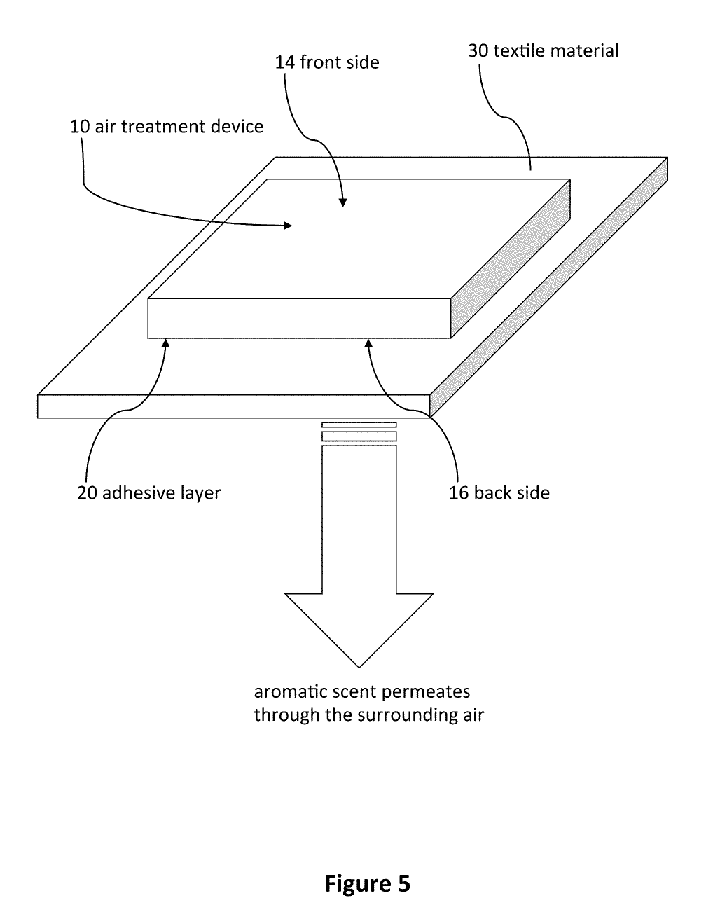

FIG. 5 illustrates a perspective view of an air treatment device 10. The air treatment device 10 can include an adhesive layer 20 that is arranged and configured to adhere to a variety of materials, such as a textile material 30, including clothing and linens, for at least a predetermined amount of time (e.g. 12 hours).

In some embodiments, the textile material 30 comprises at least one of a plant textile, a synthetic textile, and an animal textile. For example, the plant textile can include cotton, flax, jute, hemp, modal or bamboo fiber, and pina (pineapple fiber). Ramie fibers can also be used with a blend of other fibers, such as cotton. Also, plant textiles, such as nettles, which can be used to make a fiber and fabric very similar to hemp. Alternatively, flax can also be used. Lyocell, which is a tough fabric that is often blended with other fabrics such as cotton, can also be used as a synthetic fabric derived from wood pulp and is often described as a synthetic equivalent.

As well, the textile material 30 can comprise a synthetic textile, such as polyester fiber, which is used in all types of clothing, either alone or blended with fibers such as cotton. Other examples of synthetic textiles include aramid fiber (e.g. Twaron), acrylic fiber, nylon fiber, polyactide fibers (e.g. Ingeo), and milk fibers.

Additionally, the textile material 30 can comprise an animal textile, which is commonly made from hair, fur, skin, or silk (e.g., wool and cashmere). The textile material 30 can also be comprised of clothing and/or linens such as, bed sheets, a pillow, or a pillow case. The clothing can be comprised of woven, non-woven, knitted, netted, or technical fabrics, or any other material combinations for making garment top clothing, coats, jackets and/or linens. The linens can be comprised of any other cloth or fabric type material or any other textile material combinations used for making linens and/or pillow coverings, for example pillow cases or bed sheets.

As noted above, the clothing type can be a top garment, such as a shirt, blouse, coat, jacket, or a hat. The clothing type can also include a bottom garment, such as a dress, pants, or shorts. In one example, the user may insert the air treatment device 10 into the lining or pocket of a sweatshirt during an exercise routine. The air treatment device 10 can also be inserted between a pillow and pillow case—the aromatic scent 18 dispersed from the pad 12 through the pillow case (textile material 30) can, for example, temporarily relieve nasal congestion thereby facilitating a sounder and healthier sleep. The adhesive layer 20 can even be configured to adhere to other surfaces, such as paper. For example, in some embodiments, the air treatment device 10 can be inserted into a magazine for marketing purposes, similar to scented perfume or cologne strips. The user can then remove the air treatment device 10 from the magazine and adhere said air treatment device 10 inside of a shirt or jacket.

As well, the air treatment device 10 can include an aromatic scent 18. In some embodiments, the aromatic scent 18 can be arranged and configured to penetrate through the pad 12 and substrate, such as textile material 30, whereby the aromatic scent 18 permeates through the surrounding air.

The chemical state of the aromatic scent 18, which may comprise a combination of different aromatic scents, scented essential oils or extracts or chemically compounded scented fragrances, are not limited to one state and can be in the form of a gel, liquid, paste, or powder. For example, basil oil, which is used in perfumes and aromatherapy, can be combined with the decongestive properties of eucalyptus and menthol oils. The aromatic scent 18 can comprise a combination of these three oils and can, for example, be used as a sleep aid or help to temporarily relieve nasal congestion or to alleviate flu symptoms.

In some embodiments, the aromatic scent 18 defines a scent type comprising coffee. Research indicates that smelling coffee may offer its own health benefits, apart from drinking coffee. See Kelley Colihan, Coffee's Aroma Enough to Stir Brain? http://www.webmd.com/brain/news/20080313/coffees-aroma-stirs-the-brain. According to recent findings, the mere aroma of coffee can stimulate the brain and make one more alert, a useful benefit for people who have trouble waking up in the morning. Coffee's aroma has also been shown to have a calming effect on stress or an antioxidant function. Accordingly, the air treatment device 10 that emits a coffee scented aromatic 18 can either be worn during the day or inserted between a pillow and pillowcase, on a bedspread, or bed sheets (e.g. textile material 30) to help one get out of bed in the morning.

Additionally, the aromatic scent 18 can define a variety of other scent types, such as menthol, eucalyptus, peppermint, lavender, camphor, jasmine, sandalwood, lilac, lily, chamomile, vanilla, tea tree, ylang ylang, rose, clove, cedar wood, bergamot, clementine, frankincense, lemon, marjoram, patchouli, rosemary, blue cypress, and cocoa. Importantly, the aromatic scent can also include a combination of the above scent types, scented essential oils or extracts, or chemically compounded scented fragrances.

The referenced aromatic scents 18 have healing and therapeutic properties that can be specifically tailored to one's particular mental/physical constitution. As noted above, a coffee scent can energize, uplift and help with stress, anxiety and depression. Menthol aromatic scent can assist in opening nasal passages and improving breathing—this scent can be used to relieve nasal congestion to improve one's breathing during sleep so that one can wake up feeling refreshed and energized.

The eucalyptus scent can reduce respiratory issues, act as a decongestant, and can mitigate migraine pain. A peppermint scent can help with respiratory problems, headaches, nausea, and fevers, as well as enhancing one's mood and sharpening focus. Lavender scent can act as a stress reliever and an antidepressant, decongestant, sedative, and migraine reliever. Lavender can also reduce agitation and help with insomnia. Camphor can alleviate coughing. Jasmine can act as an antidepressant and reduce respiratory conditions. Sandalwood is a relaxing agent and that can provide tension relief. Lilac and lily scents are calming agents and mood enhancers. Vanilla scents can have a relaxing and calming effect.

Furthermore, a tea tree scent can aid respiratory conditions, whereas a ylang-ylang scent can act as a calming agent and help with headaches and nausea. A rose scent can act as an antidepressant, reduce anxiety, and alleviate asthma. A clove scent can ease respiratory problems, such as coughs, colds, sinusitis and asthma. Cedar wood scent can alleviate stress and anxiety, as well as act as a calming agent. Bergamot scent can alleviate stress, depression and anxiety. Clementine scent can help with insomnia, depression, and stress. A frankincense aromatic scent can help with relaxation, depression, and increase spiritual awareness. A lemon aromatic scent can act as a mood enhancer, and help to alleviate headaches and fever. A marjoram aromatic scent can help with anxiety and stress relief, as well as to combat fatigue and depression. A patchouli aromatic scent can help to relieve anxiety, depression, and fatigue. A rosemary aromatic scent can help with depression, alleviate migraines, and act as a mental stimulant. A blue cypress aromatic scent can promote a sense of clarity and focus; and, a cocoa aromatic scent can enhance feelings of serenity, calm, and relaxation. As noted above, the referenced aromatic scents 18 can be used alone or in combination with each other, or any additional aromatic scents 18, scented essential oils or extracts, or chemically compounded scented fragrances.

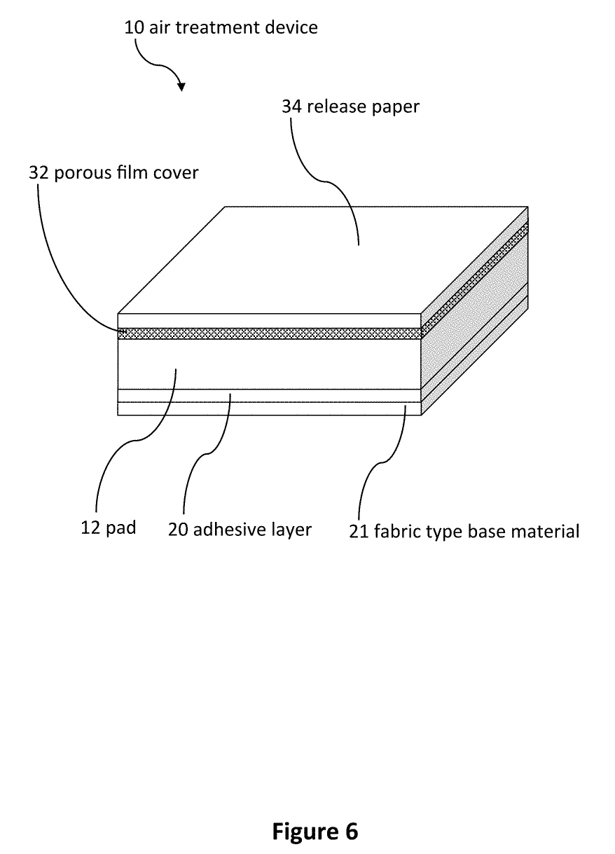

FIG. 6 illustrates a cross-sectional view of an air treatment device 10. The air treatment device 10 can include a porous film cover 32 attached to the front side of the pad 12. The porous film cover 32 can be arranged and configured to seal at least a portion of the aromatic scent 18 infused within the pad 12. The porous film cover 32, which seals the aromatic substance within the pad 12, can be composed of a micro-porous flexible hypoallergenic fabric or plastic type backing. The porous film cover 32 can also be composed of perforated plastic or wax paper.

The air treatment device 10 can include a release paper 34 removably attached above the porous film cover 32. It should be appreciated that even though the release paper 34 is attached above the porous film cover 32, the release paper 34 may be adhered to the air treatment device 10 via the adhesive layer 20. The release paper 34 can be arranged and configured to protect the adhesive layer 20 to thereby preserve an ability of the adhesive layer 20 to adhere to the textile material 30. Finally, as shown in FIG. 6, the adhesive layer 20 can be disposed over the fabric type base material 21, which can form the base of the air treatment device 10.

Moreover, the pad 12 can comprise at least one of micro-porous flexible hypoallergenic fabric and plastic, such as certain cottons that are not chemically treated (so-called “green fabrics”) and have germ-repelling properties. Other “green fabrics” that are less prone to causing an allergic reaction can also compose the pad 12, such as linen, cotton, and hemp.

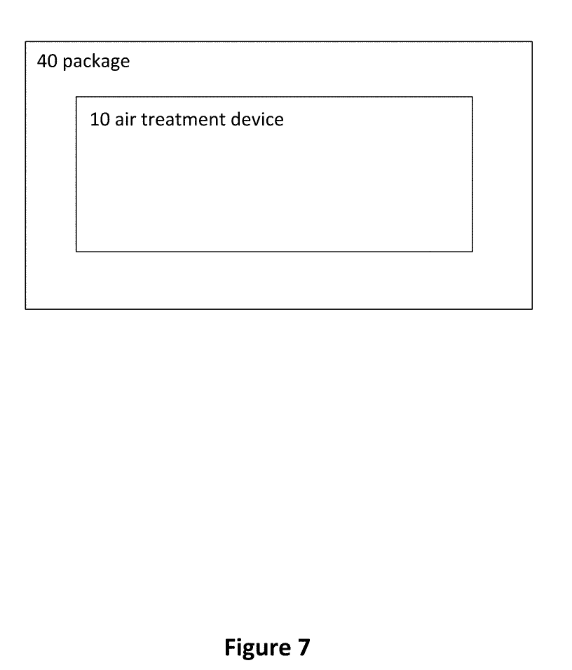

FIG. 7 illustrates a view of an air treatment device 10 enclosed within a package 40. The package 40 can be comprised of foil, plastic, paper, and/or metal. In some embodiments, the package 40 fully or partially encloses the air treatment device 10.

### Air Treatment Systems and Kit Embodiments

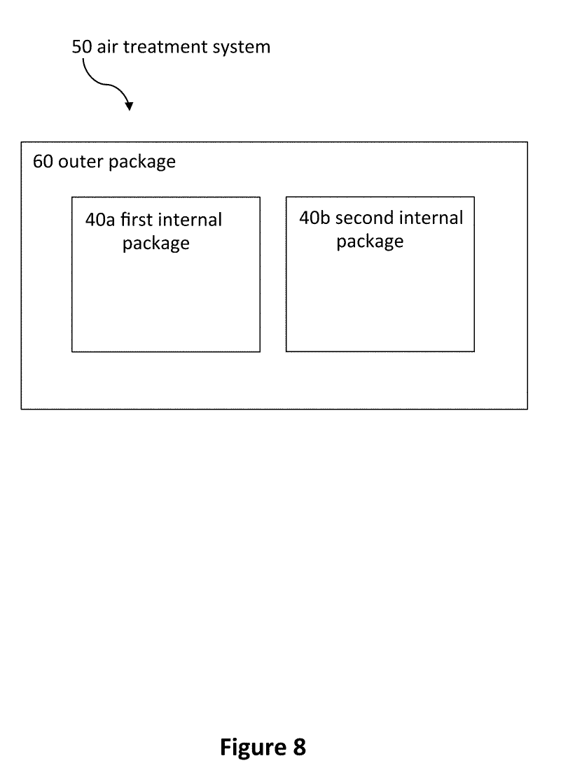

FIG. 8 illustrates an air treatment system 50 comprising an outer package 60 that encloses the first internal package 40a and the second internal package 40b. In some embodiments, the outer package 60 can be composed of foil, plastic, paper, and/or metal. The air treatment system 50 can include a first scented device 10a including a first pad 12a having a front side 14a and a back side 16a that faces opposite the front side 14a. The air treatment system 50 can include a first aromatic scent 18a infused within the first pad 12a, a first adhesive layer 20a disposed along the back side 16a of the first pad 12a, a first release paper 34a removably attached to the first adhesive layer 20a, a first porous film cover 32a attached to the front side 14a of the first pad 12a, and a first internal package 40a that encloses the first scented device 10a. The first adhesive layer 20a can be arranged and configured to adhere to a textile material 30 for at least a predetermined amount of time.

Additionally, the air treatment system 50 can include a second scented device 10b with a second pad 12b having a front side 14b and a back side 16b that faces opposite the front side 14b. Also, the air treatment system 50 can include a second aromatic scent 18b infused within the second pad 12b, a second adhesive layer 20b disposed along the back side 16b of the second pad 12b, and a second release paper 34b removably attached to the second adhesive layer 20b. The air treatment system 50 can also include a second porous film cover 32b attached to the front side 14b of the second pad 12b, and a second internal package 40b that encloses the second scented device 10b. The second adhesive layer 20b is arranged and configured to adhere to the textile material 30 for at least the predetermined amount of time.

In some embodiments, the first aromatic scent 18a and the second aromatic scent 18b are the same. For example, the outer package 60 may contain twelve internal packages 40, which in turn may contain twelve respective air treatment devices 10, each of which has a coffee scent.

Alternatively, the first aromatic scent 18a and the second aromatic scent 18b can have different scents. For example, the outer package 60 may contain both a first internal package 40a containing an air treatment device 10a having a lavender scent and a second internal package 40b containing an air treatment device 10b having a eucalyptus scent. In this regard, the outer package 60 may be a “variety pack,” containing unique air treatment devices 10, each having its own scent.

### Interpretation

None of the structures and/or devices described herein is essential or indispensable. Any of the structures and/or devices can be adjusted or modified. Other or additional structures and/or devices can be used. Any portion of any of the structures and/or devices disclosed or illustrated in one embodiment or example in this specification can be combined or used with or instead of any other portion of any of the structures, and/or devices disclosed or illustrated in a different embodiment or example. The embodiments and examples provided herein are not intended to be discrete and separate from each other.

The section headings and subheadings provided herein are non-limiting. The section headings and subheadings do not represent or limit the full scope of the embodiments described in the sections to which the headings and subheadings pertain. For example, a section titled “Topic 1” may include embodiments that do not pertain to Topic 1 and embodiments described in other sections may apply to and be combined with embodiments described within the “Topic 1” section.

The various features and processes described above may be used independently of one another, or may be combined in various ways. All possible combinations and subcombinations are intended to fall within the scope of this disclosure. In addition, certain method, event, state, or process blocks may be omitted in some implementations. The methods, steps, and processes described herein are also not limited to any particular sequence, and the blocks, steps, or states relating thereto can be performed in other sequences that are appropriate. For example, described tasks or events may be performed in an order other than the order specifically disclosed. Multiple steps may be combined in a single block or state. The example tasks or events may be performed in serial, in parallel, or in some other manner. Tasks or events may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require at least one of X, at least one of Y, and at least one of Z to each be present.

The term “and/or” means that “and” applies to some embodiments and “or” applies to some embodiments. Thus, A, B, and/or C can be replaced with A, B, and C written in one sentence and A, B, or C written in another sentence. A, B, and/or C means that some embodiments can include A and B, some embodiments can include A and C, some embodiments can include B and C, some embodiments can only include A, some embodiments can include only B, some embodiments can include only C, and some embodiments include A, B, and C. The term “and/or” is used to avoid unnecessary redundancy.

While certain example embodiments have been described, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions, and changes in the form of the methods and systems described herein may be made without departing from the spirit of the inventions disclosed herein.

The following is claimed:

1. An air treatment device, comprising:
a pad having a front side and a back side that faces opposite the front side;
an aromatic scent infused within the pad; and
an adhesive layer disposed along the back side of the pad.

2. The air treatment device of claim 1, wherein the adhesive layer is arranged and configured to adhere to a textile material for at least a predetermined amount of time.

3. The air treatment device of claim 2, wherein the predetermined time is at least 12 hours.

4. The air treatment device of claim 2, wherein the aromatic scent is arranged and configured to penetrate through the pad and textile material whereby the aromatic scent permeates through the surrounding air.

5. The air treatment device of claim 2, further comprising a porous film cover attached to the front side of the pad, wherein the porous film cover is arranged and configured to seal at least a portion of the aromatic scent infused within the pad.

6. The air treatment device of claim 5, further comprising a release paper removably attached to the adhesive layer, wherein the release paper is arranged and configured to protect the adhesive layer to thereby preserve an ability of the adhesive layer to adhere to the textile material.

7. The air treatment device of claim 6, wherein the air treatment device is enclosed within a package.

8. The air treatment device of claim 7, wherein the outer package comprises at least one of foil and plastic.

9. The air treatment device of claim 2, wherein the textile material comprises at least one of a plant textile, a synthetic textile, and an animal textile.

10. The air treatment device of claim 9, wherein the textile material comprises one of clothing and/or linens such as, bed sheets, a pillow, and a pillow case.

11. The air treatment device of claim 2, wherein the pad comprises at least one of micro-porous flexible hypoallergenic fabric and plastic.

12. The air treatment device of claim 11, wherein the adhesive layer comprises a latex-free acrylic adhesive.

13. The air treatment device of claim 2, wherein the aromatic scent defines a chemical state comprising at least one of a gel, liquid, paste, and powder.

14. The air treatment device of claim 13, wherein the aromatic scent defines a scent type comprising coffee.

15. The air treatment device of claim 13, wherein the aromatic scent defines a scent type comprising at least one of menthol, eucalyptus, peppermint, lavender, camphor, jasmine, sandalwood, lilac, lily, chamomile, vanilla, tea tree, ylang ylang, rose, clove, cedar wood, bergamot, clementine, frankincense, lemon, marjoram, patchouli, rosemary, blue cypress, and cocoa.

16. An air treatment system, comprising:
a first scented device including a first pad having a front side and a back side that faces opposite the front side, a first aromatic scent infused within the first pad, a first adhesive layer disposed along the back side of the first pad, a first release paper removably attached to the first adhesive layer, a first porous film cover attached to the front side of the first pad, and a first internal package that encloses the first scented device, wherein the first adhesive layer is arranged and configured to adhere to a textile material for at least a predetermined amount of time; and
a second scented device including a second pad having a front side and a back side that faces opposite the front side, a second aromatic scent infused within the second pad, a second adhesive layer disposed along the back side of the second pad, a second release paper removably attached to the second adhesive layer, a second porous film cover attached to the front side of the second pad, and a second internal package that encloses the second scented device, wherein the second adhesive layer is arranged and configured to adhere to the textile material for at least the predetermined amount of time.

17. The air treatment system of claim 16, further comprising an outer package that encloses the first internal package and the second internal package.

18. The air treatment system of claim 17, wherein the first aromatic scent and the second aromatic scent are the same.

19. The air treatment system of claim 17, wherein the first aromatic scent and the second aromatic scent are different.
